(19) United States
(12) Patent Application Publication
(10) Pub. No.: US 2013/0218219 A1
(43) Pub. Date: Aug. 22, 2013

(54) Pulse Parameters And Electrode Conffigurations For Reducing Patient Discomfort From Defibrillation

(71) Applicants: Avi Allon Livnat, Tel Aviv-Yafo (IL); Lazaro Salomon Azar, Givatayim (IL)

(72) Inventors: Avi Allon Livnat, Tel Aviv-Yafo (IL); Lazaro Salomon Azar, Givatayim (IL)

(73) Assignee: SMARTWAVE MEDICAL LTD., Tel-Aviv (unknown)

(21) Appl. No.: 13/818,243

(22) PCT Filed: Aug. 22, 2011
(86) PCT No.: PCT/US2011/048574

§ 371 Date: May 7, 2013

Related U.S. Application Data
(60) Provisional application No. 61/402,009, filed on Aug. 23, 2010.

(51) Int. Cl. A61N 1/39

(52) U.S. Cl. CPC A61N 1/3987
USPC 607/6

(57) ABSTRACT

Devices, systems and methods for reducing patent discomfort during defibrillation by synchronizing defibrillation pulse delivery with a patient breathing cycle are described. Embodiments provide for a defibrillator having at least one electrode lead with one or more electrodes, a controller for determining whether fibrillation exists, a voltage generator for producing and discharging one or more electrical pulses to the electrode lead system and at least one breathing sensor for collecting and transmitting information relating to the breathing cycle of the patient to the controller. The controller may process the information from the breathing sensor, determine when one or more phases or instants of the breathing cycle are occurring and emit a command signal to the voltage generator to discharge defibrillation pulses to the electrode lead system in synchronization with the one or more phases or instants of the breathing cycle.

### CROSS-REFERENCE TO RELATED APPLICATIONS

The present application claims priority to U.S. Provisional Patent Application No. 61/402,009, filed on Aug. 23, 2010 and entitled “Reduced Pain Caused by Cardioversion Shocks by Applying the Shock at a Certain Phase of the Respiratory Cycle,” the disclosure of which is incorporated herein by reference in its entirety.

### FIELD

Devices, systems and methods relating to atrial defibrillation are described herein. Embodiments of the present disclosure more specifically reduce patient discomfort during defibrillation by delivering one or more electrical defibrillation pulses to electrodes positioned in or around the heart in synchronization with one or more phases or instants of the breathing cycle of a patient.

### BACKGROUND

Atrial Fibrillation (“AF”) is the most common cardiac arrhythmia involving at least one of the left or right atrium of the heart. One way to defibrillate an atrium is by delivering electrical defibrillation pulses to the heart at specific times during the cardiac cycle. Systems and devices for delivering these pulses may be external to and/or implanted within the body. Atrial defibrillation using an implantable atrial defibrillator generally includes automatically detecting AF and automatically delivering one or more electrical defibrillation pulses to the left and/or right atrium of the heart. Delivering an electrical pulse may be intolerably painful for a conscious patient and discourage the use of automatic implantable atrial defibrillators, particularly when high-energy pulses are delivered. However, delivering an electrical pulse having an energy that is too low will result in an unsuccessful defibrillation attempt. Atrial defibrillation should therefore be tolerable, effective and reduce patient discomfort.

The pain associated with electrical defibrillation pulses delivered to the heart is thought to be caused by hyper contraction of skeletal muscle located primarily in and around the chest region and/or direct stimulation of the nerves in this area of the body. It has been shown that the hyper contraction of skeletal muscles caused by an electrical pulse is not caused by direct stimulation of the skeletal muscle, but rather by direct stimulation of the nerves that innervate the muscle. Furthermore, it is known that administering a drug to block neuromuscular junctions in the muscles may result in near total suppression of skeletal muscle contractions when electrical pulses are delivered.

General discussions of defibrillation and resulting skeletal muscle activation are provided in Murgatroyd F. D., Slade A. K., Sopher S. M., Rowland E., Ward D. E., Camm A. J., Efficacy and Tolerability of Transvenous Low Energy Cardioversion of Paroxysmal Atrial Fibrillation in Humans, J. AM. COLL CARDIOL., 25:1347-53 (1995), Jayam V., Zviman M., Jayanti V., Roguin A., Halperin H., Berger R. D., Internal Defibrillation with Minimal Skeletal Muscle Activation: A New Paradigm Toward Painless Defibrillation, HEART RHYTHM, 2:1108-13 (2005) and Sweeney J. D., Skeletal Muscle Response to Electrical Stimulation, ELECTRICAL STIMULATION AND ELECTROPATHOLOGY, p. 293 (Cambridge University Press 1992).

### SUMMARY

Some embodiments described herein are directed to a defibrillator for defibrillating the heart of a patient that includes an electrode lead system having at least one electrode lead with one or more electrodes, a controller configured to determine whether a heart is fibrillating and emit a command signal if fibrillation exists, a voltage generator in communication with the controller and the electrode lead system that produces and discharges one or more electrical pulses to the electrode lead system after receiving the command signal and at least one breathing sensor in communication with the controller and configured that collects and transmits information relating to a breathing cycle of the patient to the controller. In some embodiments, the controller processes the information received from the at least one breathing sensor, determines when one or more phases or instants of the breathing cycle of the patient are occurring and emits a command signal to the voltage generator to produce and discharge one or more electrical pulses to the electrode lead system in synchronization with the one or more phases or instants of the breathing cycle of the patient.

In some embodiments, the defibrillator may be positioned outside the patient and the electrode lead system may include at least one external defibrillation electrode positioned outside the patient, at least one internal electrode lead positioned partially in or around the heart of the patient and/or at least one internal electrode lead positioned partially in or around the heart of the patient and at least three external defibrillation electrodes positioned outside the patient.

Some device embodiments of the present disclosure may be configured with the at least one breathing sensor positioned on the chest of the patient and having an electromechanical sensor for detecting chest expansion. The at least one breathing sensor may communicate with the defibrillator using wireless means according to some embodiments. The at least one breathing sensor may be configured, in some embodiments, to collect and transmit information relating to the impedance between at least one electrode of the electrode lead system positioned partially in or around the heart of the patient and at least one electrode of the electrode lead system positioned outside the patient. The at least one electrode of the electrode lead system positioned outside the patient may be the defibrillator itself. In some embodiments, the at least one breathing sensor may collect and transmit information relating to the impedance between at least one electrode of the electrode lead system positioned partially in or around the heart of the patient and at least one electrode of the electrode lead system positioned outside the patient using a low-voltage, high-frequency signal. In some embodiments, the at least one breathing sensor may include one or more accelerometers contained within the defibrillator, external to the body of the defibrillator and/or contained within one or more of the at least one electrode lead.

In some embodiments of the defibrillator according to the present disclosure, the one or more electrical pulses may be delivered by the voltage generator to the electrode lead system as a pulse train of one or more sequential electrical pulses. The pulse train may consist of up to 12 pulses. In some embodiments, the defibrillator may include a controller that emits a command signal to the voltage generator to produce and discharge one or more electrical pulses to the electrode lead system at the peak of the inspiratory phase of the breathing cycle of the patient.

In some embodiments, the defibrillator may be subcutaneously implanted within the patient. The defibrillator may also be an atrial defibrillator for defibrillating the atria. Some embodiments may include an electrode lead system that has at least one electrode for sensing atrial fibrillation.

In some embodiments, the at least one breathing sensor may include one or more strain gauges for detecting and measuring movement of the at least one electrode lead caused by breathing action of the patient.

In some embodiments, the defibrillator may analyze variations in the heart beat of the patient to synchronize the delivery of one or more electrical pulses with one or more phases or instants of the breathing cycle of the patient. For example, the at least one breathing sensor may be configured to sense activity of the phrenic nerves of the patient to synchronize the delivery of one or more electrical pulses with one or more phases or instants of the breathing cycle of the patient. In some embodiments, the voltage generator may produce and discharge one or more electrical pulses to the electrode lead system in synchronization with the expiratory phase of the breathing cycle of the patient. In some embodiments, the voltage generator may produce and discharge one or more electrical pulses to the electrode lead system in synchronization with the expiratory phase of the breathing cycle of the patient and immediately after an R-wave of the heart of the patient. In some embodiments, the voltage generator may produce and discharge one or more electrical pulses to the electrode lead system in synchronization with the inspiratory phase of the breathing cycle of the patient. In some embodiments, the voltage generator may produce and discharge one or more electrical pulses to the electrode lead system in synchronization with the inspiratory phase of the breathing cycle of the patient and immediately after an R-wave of the heart of the patient. In some embodiments, the voltage generator may produce and discharge one or more electrical pulses to the electrode lead system during the inhibitory period of the phrenic nerve of the patient based on the information received from the at least one breathing sensor relating to the breathing cycle of the patient.

In some embodiments, the at least one breathing sensor may be activated only when fibrillation has been detected and synchronization of one or more electrical pulses with the breathing cycle of the patient is required. The defibrillator may automatically produce and deliver ventricular defibrillation pulses to the heart upon detecting ventricular fibrillation, according to some embodiments.

Some embodiments of the present disclosure may be directed to methods for defibrillating the heart of a patient with a defibrillator. Such methods may include positioning in or around the heart of the patient an electrode lead system having at least one electrode lead with one or more electrodes, monitoring cardiac activity of the heart to determine whether the heart is fibrillating, sending a command signal to a voltage generator indicating that the heart is fibrillating (if the heart is fibrillating), activating at least one breathing sensor to monitor a breathing cycle of the patient and collect information relating to the breathing cycle, determining, based on the information relating to the breathing cycle, a time for delivering at least one defibrillation pulse to the heart of the patient in synchronization with one or more phases or instants of the breathing cycle of the patient, generating at the voltage generator the at least one defibrillation pulse and/or delivering the at least one defibrillation pulse to the heart in synchronization with one or more phases or instants of the breathing cycle. In some method embodiments, the at least one defibrillation pulse may be automatically delivered to the heart without synchronization with one or more phases or instants of the breathing cycle when ventricular fibrillation is detected.

In some embodiments, the activating step may trigger the charging of one or more high-voltage capacitors in the defibrillator. According to some embodiments, after one or more high-voltage capacitors are fully charged, monitoring of the cardiac activity of the heart may continue to determine whether the heart is still fibrillating. If no fibrillation is detected during the continued monitoring of the cardiac activity of the heart, such monitoring may continue for a preset amount of time. In some embodiments, the high-voltage capacitors may be discharged, the breathing sensors may be deactivated and normal monitoring of the cardiac activity of the heart may resume if no fibrillation is detected during the preset amount of time.

In some embodiments, the delivering step may include delivering the at least one defibrillation pulse to the heart at the peak of the inspiratory phase of the breathing cycle of the patient. In some embodiments, the delivering step may include delivering the at least one defibrillation pulse to the heart in synchronization with the expiratory phase of the breathing cycle of the patient. In some embodiments, the delivering step may include delivering the at least one defibrillation pulse to the heart in synchronization with the expiratory phase of the breathing cycle of the patient and immediately after an R-wave of the heart of the patient. In some embodiments, the delivering step may include delivering the at least one defibrillation pulse to the heart in synchronization with the inspiratory phase of the breathing cycle of the patient. In some embodiments, the delivering step may include delivering the at least one defibrillation pulse to the heart in synchronization with the inspiratory phase of the breathing cycle of the patient and immediately after an R-wave of the heart of the patient. In some embodiments, the delivering step may include delivering the at least one defibrillation pulse to the heart during the inhibitory period of the phrenic nerve of the patient the information relating to the breathing cycle of the patient.

Some embodiments of the present disclosure may be directed to a heart defibrillation system that includes a defibrillator configured to be implanted within a patient and a communication device disposed outside the patient and configured to communicate with the defibrillator. The defibrillator may include an electrode lead system having at least one electrode lead with one or more electrodes, a controller configured to determine whether a heart is fibrillating and emit a command signal if fibrillation exists, a voltage generator in communication with the controller and the electrode lead system to produce and discharge one or more electrical pulses to the electrode lead system after receiving the command signal and at least one breathing sensor in communication with the controller and configured to collect and transmit information relating to a breathing cycle of the patient to the controller. In some embodiments, the controller may process the information received from the at least one breathing sensor, determine when one or more phases or instants of the breathing cycle of the patient are occurring and emit a command signal to the voltage generator to produce and discharge one or more electrical pulses to the electrode lead system in synchronization with the one or more phases or instants of the breathing cycle of the patient.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE FIGURES

FIG. 1 shows an external defibrillator having electrodes implanted within a patient according to some embodiments of the present disclosure.

FIG. 2 shows a defibrillator implanted within a patient according to some embodiments of the present disclosure.

FIG. 3 shows a block diagram of an implantable atrial defibrillator according to some embodiments of the present disclosure.

FIG. 4 shows a defibrillation system according to some embodiments of the present disclosure.

FIG. 5 shows a timing sequence for synchronizing one or more electrical defibrillation pulses with the breathing cycle of a patient according to some embodiments of the present disclosure.

FIG. 6 shows a flow diagram of a method for synchronizing one or more electrical defibrillation pulses with the breathing cycle of a patient according to some embodiments of the present disclosure.

Like reference symbols in the various drawings indicate like elements.

### DETAILED DESCRIPTION

The subject matter described herein relates to defibrillating the heart using an implantable atrial defibrillation system and is not limited in its application to the details set forth in the following disclosure or exemplified by the illustrative embodiments. The subject matter is capable of other embodiments and of being practiced or carried out in various ways. Features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination or any other described embodiment of the present disclosure. Certain features described in the context of various embodiments are not to be considered essential features of those embodiments, unless the embodiment is inoperative without those elements.

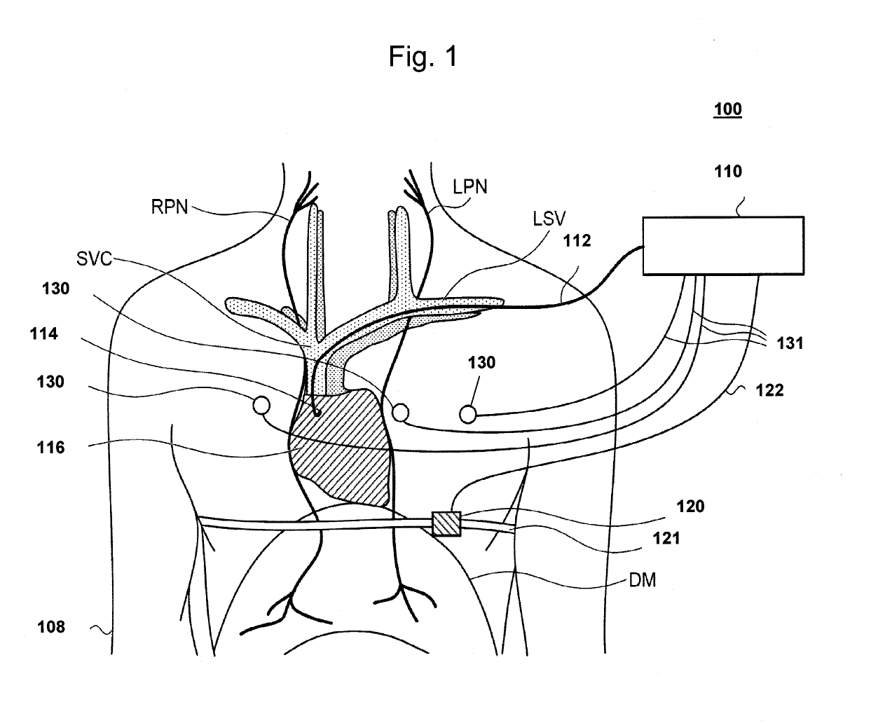

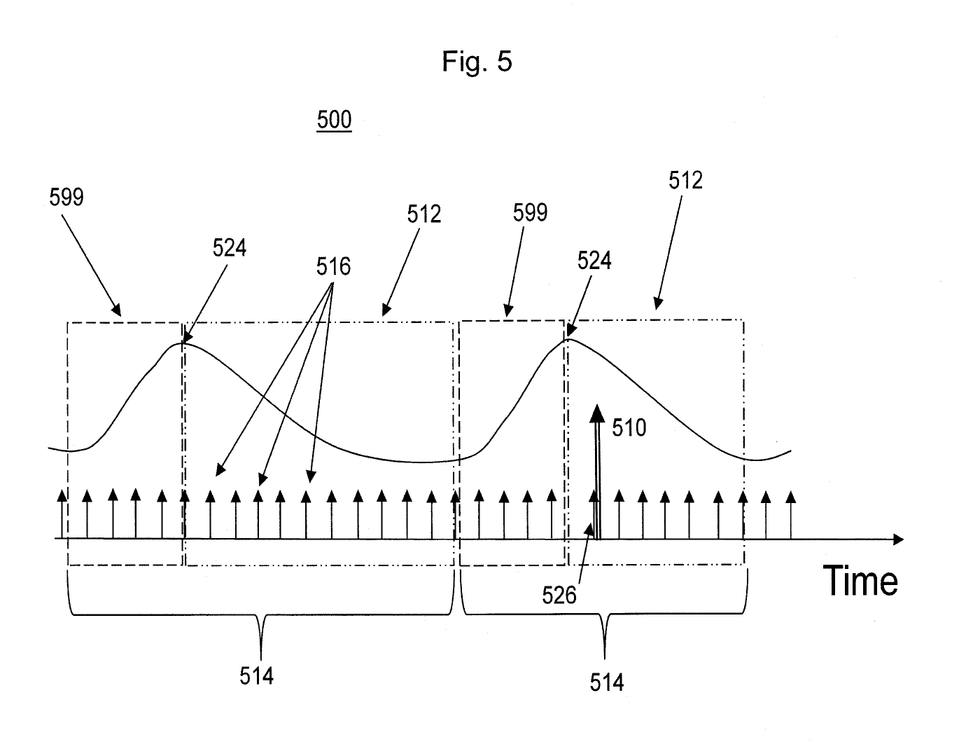

The present disclosure is directed more specifically to devices, systems and methods of atrial defibrillation for reducing pain associated therewith by applying one or more electrical defibrillation pulses in or around the heart in synchronization with a specific phase or instant of the breathing cycle of a patient. In some embodiments, one or more electrical defibrillation pulses may be delivered to the heart at or around the time of peak inspiration (see 524 in FIG. 5) when a patient has just completed inhaling air into the lungs. During the breathing cycle, the depolarization of nerves in and around the area of the lungs, such as for example the left phrenic nerve (LPN) and the right phrenic nerve (RPN) (see FIG. 1) and other nerves that innervate the intercostal muscles, the diaphragm muscles and other chest muscles, occurs at peak inspiration (see 524 in FIG. 5) and/or shortly before the mechanical chest extension peak. Using the depolarization of the nerves at peak inspiration as a reference point, it is possible to deliver one or more electrical pulses to the heart following peak inspiration during the refractory period of the nerves. Because the nerves cannot respond to stimuli during this refractory state, it is possible to deliver electrical defibrillation pulses to the heart at this time without stimulating the surrounding nerves and causing the pain typically associated with such pulses.

Furthermore, the left and right phrenic nerves, which together constitute the main nerve trunk that innervates the chest and the respiratory muscles, have known inhibitory periods that occur immediately after peak inspiration. As part of the neural control of the breathing cycle, firing of certain motor neurons of the phrenic nerves during the inhibitory period, actually prevents the chest and the respiratory (diaphragm) muscles from contracting. See Richter, D. W., Generation and Maintenance of the Respiratory Rhythm, J. EXP. BIOL. 93-107 (1982). Thus, delivering electrical defibrillation pulses during the inhibitory period of the right phrenic nerve (RPN) and/or left phrenic nerve (LPN) may also significantly reduce muscle movement during defibrillation and the pain associated therewith.

Delivering one or more electrical defibrillation pulses in synchronization with the peak inspiration (see 524 in FIG. 5) of the breathing cycle may also reduce discomfort because at this time the lungs are filled with air, which increases the impedance between the heart and the chest muscles (e.g., intercostal muscles and/or the diaphragm) and decreases the magnitude of the electrical defibrillation pulses felt by the nerves in those muscles.

FIG. 1 shows a defibrillation system (100) that includes an external defibrillator (110) with an electrode lead (112) having one or more electrodes (114) implanted within the heart (116) of a patient (108) according to some embodiments of the present disclosure. FIG. 1 also shows the right phrenic nerve (RPN), left phrenic nerve (LPN), left subclavian vein (LSV), superior vena cava (SVC) and diaphragm muscle (DM). The electrode lead (112) may be connected to the external defibrillator (110) and enter the heart (116) through the LSV and SVC as shown in FIG. 1. In some embodiments, one or more external defibrillation electrodes (130) may be used in addition to or instead of the lead electrode (112). The one or more external defibrillation electrodes (130) may be connected to the external defibrillator (110) using one or more wires (131).

Embodiments of the system (100) according to the present disclosure may include a breathing sensor (120) for monitoring, detecting and/or measuring phases and/or instants of the breathing cycle of the patient (108) and transmitting signals and/or data representative of information relating to such detection and measurement to the external defibrillator (110). The breathing sensor (120) may capable of communicating with the external defibrillator (110) using a communication link (122). The communication link (122) may be a physical wire or, in some embodiments, a wireless communication transmission path with short-range and/or long-range capabilities. In some embodiments, the communication link (122) may be an ultrasonic link communicating with an external device in contact with the body of the patient (108). In some embodiments, the communication link (122) may be a short-range radio frequency (“RF”) communication link and may use a proprietary protocol for communicating with the external defibrillator (110). The breathing sensor (120) may be a dedicated sensor, such as a belt (121) placed around the chest of the patient (108) and having an electromechanical sensor for sensing chest expansion, as shown in FIG. 1.

In some embodiments, the breathing sensor (120) may monitor, detect and/or measure rhythmic changes in lung impedance, for example, between at least one of the one or more electrodes (114) and the one or more external defibrillation electrodes (130). In some embodiments, the breathing sensor (120) may monitor, detect and/or measure rhythmic changes in lung impedance between at least one of the one or more electrodes (114) and the external defibrillator (110) itself when the defibrillator (110) is placed in contact with the patient (108). In some embodiments, rhythmic changes in the breathing cycle of the patient (108) may be monitored, detected and/or measured using one or more accelerometers contained within the external defibrillator (110) when the defibrillator (110) is placed in contact with the patient (108) or using an accelerometer (not shown) placed on the chest of the patient (108).

In some embodiments, signals and/or data representative of the breathing cycle of the patient (108) may be obtained by measuring impedance between the one or more electrodes (130) using a low-voltage, high-frequency (e.g., ˜40 kHz) signal. While FIG. 1 shows three external defibrillation electrodes (130), any number and configuration of the one or more external defibrillation electrodes (130) on the patient (108) may be suitable and in accordance with the present disclosure. In some embodiments, the function of measuring the impedance may be integrated within the external defibrillator (110).

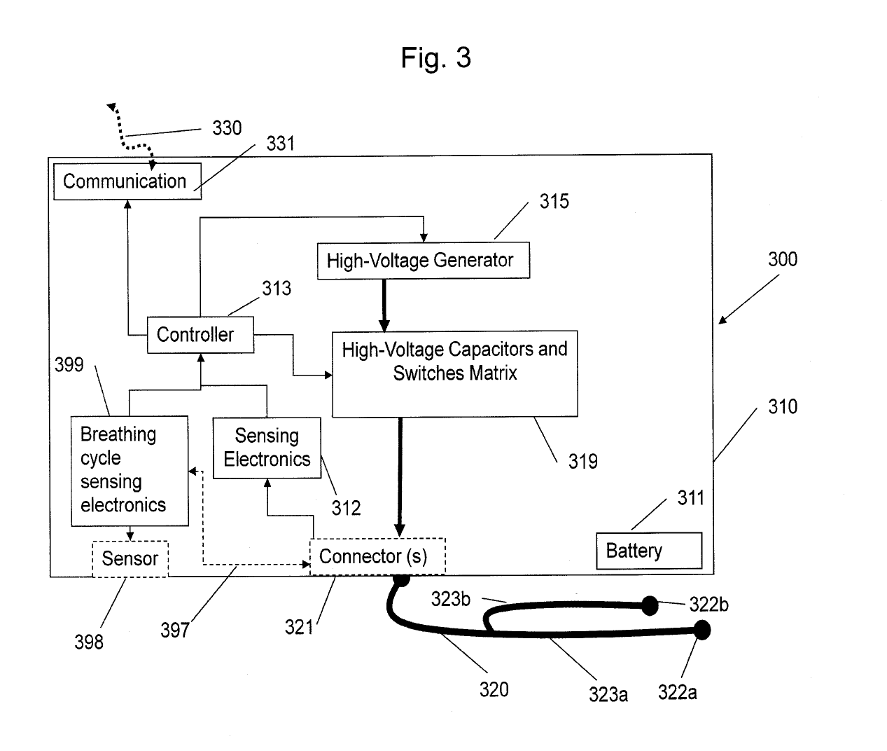

The signals and/or data representative of the breathing cycle may be transmitted to a controller (see, e.g., controller (313) in FIG. 3) of the external defibrillator (110) to analyze the breathing cycle data and determine when certain phases (e.g., inspiration and/or expiration) or instants (e.g., peak inspiration) in the breathing cycle occur. To this end, the controller may cause pulse-generating circuitry (see, e.g., high-voltage generator (315) and high-voltage capacitor and switches matrix (319) in FIG. 3) to produce one or more high-voltage, short-duration electrical defibrillation pulses to the atria and/or ventricles of the heart (116) in synchronization with a phase or instant (e.g., peak inspiration) of the breathing cycle of the patient (108). In some embodiments, a synchronization function may be housed in a separate unit that provides a trigger or enabling signal to the external defibrillator (110). In some embodiments, at least one of the one or more external defibrillation electrodes (130) may be used to deliver one or more electrical defibrillation pulses to the atria and/or ventricles at one or more different phases or instants of the breathing cycle. For example, according to some embodiments, electrical defibrillation pulses may be delivered when the lungs are fully deflated and electrical impedance between the one or more external defibrillation electrodes (130) and the heart (116) may be reduced.

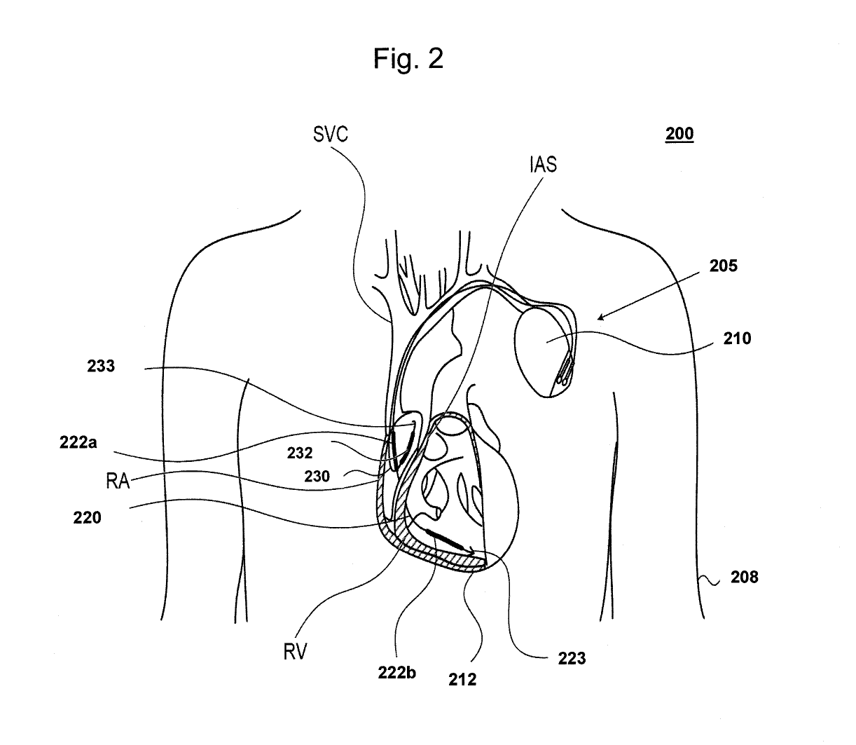

FIG. 2 shows a defibrillation system (200) that includes a subcutaneously implanted atrial defibrillator (“IAD”) (205) having a main body (210) with one or more electrode leads (e.g., 220, 230 in FIG. 2) connected thereto and positioned in and around the heart (212) of a patient (208). Each of the one or more electrode leads (e.g., 220, 230 in FIG. 2) may include at least one electrode for sensing rhythmic activity of the heart and/or delivering electrical defibrillation pulses to the heart. In some embodiments, one or more electrodes may be used for monitoring, detecting and/or measuring phases and/or instants of the breathing cycle of the patient (208). For efficient defibrillation of the left and/or right atrium with minimal discomfort to patient (208), the electrode leads (e.g., 220, 230 in FIG. 2) and associated electrodes may be located near the atria and, in some embodiments, arranged such that one or more electric fields produced by the delivered electrical defibrillation pulses is confined substantially to the targeted tissue. Exemplary electrode configurations and embodiments directed to confining electric fields to target tissue areas are disclosed in co-owned, co-pending International Patent Application No. PCT/US11/041411, filed on Jun. 22, 2011 and entitled “Pulse Parameters and Electrode Configurations for Reducing Patient Discomfort from Defibrillation,” and International Patent Application No. PCT/US11/044771, filed on Jul. 21, 2011 and entitled “Improved Use of Electric Fields for Reducing Patient,” the disclosures of which are hereby incorporated by reference in their entirety.

FIG. 2 shows an embodiment having a first electrode lead (220) and a second electrode lead (230) positioned within the heart (212) and connected to the main body (210) of the IAD (205). The first electrode lead (220) may enter the right atrium (RA) and extend into the right ventricle (RV), as shown in FIG. 2. The first electrode lead (220) may be anchored (223) at the apex of heart (212). The first electrode lead (220) may include at least two electrodes (222a) and (222b) in the RA and RV, respectively. The second electrode lead (230) may enter the RA and be anchored (233) to the heart (212) to position an electrode (232) near the interatrial septum (IAS).

The subject matter of the present disclosure contemplates various embodiments for monitoring, detecting and/or measuring phases and/or instants of the breathing cycle of the patient (208) using the IAD (205). Some embodiments may utilize RF sensing methods, wherein a low-voltage signal at high frequency (e.g., ˜40 kHz) may be used to measure the impedance between one or more locations in the chest. For example, the impedance between one or more electrodes positioned on the first electrode lead (220) and/or the second electrode lead (230), such as electrode (232), in or around the heart (212) and an electrode located outside the lungs may be measured. In some embodiments, the main body (210) of the IAD (205) may be used as an electrode outside the lungs. As the patient (208) inhales, the distance between electrodes increases as air enters and expands the lungs to cause increased impedance between the heart and electrical stimuli and the nerves innervated throughout the surrounding chest muscles.

Some embodiments of the present disclosure may utilize one or more accelerometers to monitor, detect and/or measure phases and/or instants of the breathing cycle of the patient (208) using the IAD (205). For example, a first accelerometer (not shown) may be attached to the main body (210) of the IAD (205) and a second accelerometer (not shown) may be attached to or integrated into one of the first electrode lead (220) and the second electrode lead (230). In some embodiments, the signals received from the first and/or second accelerometer may be filtered to reject low frequencies caused by common motions, such as walking or bending, but detect and accept breathing movements based on differences between the signals of the two accelerometers.

In some embodiments, the distance between the main body (210) of the IAD (205) and the first electrode lead (220) and/or the second electrode lead (230) may be measured based on changes in the expansion and contraction of the lungs. This distance may be estimated by using an ultrasonic transducer contained within the IAD (205). In some embodiments, the distance to the ribs, which may provide a strong ultrasonic reflection signal, may be measured. In some embodiments, the distance between electrodes along the first electrode lead (220) and/or the second electrode lead (230) may be assessed by, for example, measuring their mutual capacitance.

Some embodiments of the present disclosure may monitor, detect and/or measure phases and/or instants of the breathing cycle of the patient (208) by detecting and measuring the bending or other movement of the first electrode lead (220) and/or the second electrode lead (230) cause by the breathing action of the patient (208). In some embodiments, measuring the relative movement of a lead positioned in or around the heart (208), such as the first electrode lead (220) and/or second electrode lead (230), may be accomplished using one or more strain gauges attached to or integrated within the lead.

Some embodiments of the present disclosure may monitor, detect and/or measure phases and/or instants of the breathing cycle of the patient (208) by directly sensing the activity of the left phrenic nerve (LPN) and/or right phrenic nerve (RPN). In some embodiments, one or more electrodes may be placed near the left phrenic nerve (LPN) and/or the right phrenic nerve (RPN) (e.g., within the circulatory system, outside the heart (212) and/or outside main veins, such as the superior vena cava and the left subclavian vein) to monitor nerve activity. These electrodes may be positioned, for example, along the first electrode lead (220) and/or second electrode lead (230) or on other separate electrode leads.

In some embodiments, to preserve battery power, breathing sensors according to the present disclosure may be activated only when fibrillation has been detected and synchronization of one or more electrical pulses with the breathing cycle is required. In some embodiments, when ventricular fibrillation and/or unconsciousness is indicated by abnormal cardiac activity and/or little to no breathing, a ventricular defibrillation pulses may be produced and delivered automatically without synchronizing the pulses with the breathing cycle and/or a rescue team may be alerted and dispatched to the patient.

FIG. 3 shows a block diagram of an implantable atrial defibrillator (“IAD”) (300) according to some embodiments of the present disclosure. The internal construction of the IAD (300) may vary depending upon the embodiment and, in some embodiments, may be an internal construction that is known in the art. Example configurations of the IAD (300) are provided in International Publication No. WO2009/108502 to Livnat et al., filed on Feb. 11, 2009 and entitled “Atrial Defibrillation Using an Implantable Defibrillation System,” the disclosure of which is incorporated herein by reference in its entirety.

For performing the defibrillation methods contemplated by the present disclosure, the IAD (300) may include a communication transceiver (331) capable of wirelessly communicating with an external device using a communication link (330). The communication link (330) may have short-range and/or long-range capabilities. The communication link (330) may be an ultrasonic link communicating with an external device in contact with a patient's body. In some embodiments, the communication link (330) may be a short-range radio frequency (“RF”) communication link and may use a proprietary protocol for communicating with an interface device. In some embodiments, the communication link (330) may use a common protocol, such as Bluetooth technology or wireless fidelity (“Wi-Fi”), wherein the external device may include mobile devices (i.e., portable devices), such as, for example, a mobile phone, media player, smart phone, Personal Digital Assistant (“PDA”) and other handheld computing devices and the like.

The IAD (300) may have a main body (310). The main body (310) may be made of one or more bio-compatible materials known in the art. The main body (310) may contain at least one battery (311) and electronic circuitry for sensing cardiac activity, processing the sensed activity to determine whether the activity is normal or indicative of a fibrillation state and delivering one or more high-voltage defibrillation pulses. In some embodiments, the IAD (300), and in particular the electronic circuitry may be configured to differentiate between atrial and ventricular fibrillations and respond accordingly based on whether the atria or ventricles of the heart are fibrillating.

Some embodiments of the main body (310) may include at least one electrical connector (321) connected to a lead (320). In some embodiments, the lead (320) may be permanently attached to the main body (310). In some embodiments, the lead (320) may be bifurcated into sub-leads (323a) and (323b) having exposed electrodes (322a) and (322b), respectively. The number of leads, sub-leads and electrodes, as well as their specific configurations, may vary depending on the embodiment. The locations of the electrodes along the leads and/or sub-leads may also vary depending on the embodiment. For example, some embodiments of the IAD (300) may position one or more electrodes in the left and/or right atrium for pacing the heart, in addition to those electrodes used for atrial defibrillation. In some embodiments, one or more additional electrodes may be positioned in the right ventricle and used for electrocardiogram (“ECG”) sensing and delivering one or more ventricular defibrillation pulses. In some embodiments, the main body (310), or parts thereof, may be used as an electrode. In some embodiments, the communication transceiver (331) may use the lead (320) as an antenna for RF communication. Some embodiments of the IAD (300) may include a dedicated antenna, for example a coil, loop or dipole antenna, located within or outside the main body (310).

At least one of the electrodes (322a) and (322b) shown in FIG. 3 may be used for sensing ECG signals for monitoring the cardiac activity of a patient implanted with the IAD (300). In some embodiments, at least one of the electrodes (322a) or (322b) may be used both for sensing ECG data and delivering defibrillation pulses or cardiac pacing. In some embodiments, at least one of the electrodes (322a) and (322b) may be dedicated to sensing ECG signals. Embodiments of the IAD (300) may include sensing electronics (312) configured to condition (e.g., amplify and/or filter) the ECG signals. In some embodiments, the sensing electronics (312) may be configured for sensing R-waves deflections in the ECG signals. The IAD (300) may include additional sensors for monitoring cardiac activity and other bodily functions. For example, the IAD (300) may include one or more thermal sensors to monitor patient body temperature, blood oxygenation sensors, microphones to monitor sound emitted from the heart and the respiratory system, breathing sensors (e.g., capacitive sensors or sensors sensing the bending of the lead (320) due to breathing) and/or other sensors known in the art. In some embodiments, the sensing electronics (312) may include an Analog-to-Digital Converter (“ADC”).

The IAD (300) may include a controller (313) for performing signal conditioning and analysis. The controller (313) may receive data indicative of cardiac activity from the sensing electronics (312) and/or one or more other sensors and may receive commands and data from the communication transceiver (331). The controller (313) may determine the state of the cardiac activity based on ECG signals and other sensor data and control pulse-generating circuitry to produce one or more defibrillation pulses when appropriate. In some embodiments, pulse-generating circuitry may include a high-voltage generator (315) and a high-voltage capacitor and switches matrix (319) configured to produce high-voltage, short-duration pulses for defibrillating the atria and/or ventricles of the heart.

Embodiments of the present disclosure may also include a breathing sensor (398) for determining the phase of the breathing cycle of a patient. The breathing sensor (398) may be positioned within the main body (310) or positioned external to the main body (310). In some embodiments, two or more breathing sensors (398) may be used. The IAD (300) may include breathing cycle sensing electronics (399) for receiving signals from the breathing sensor (398) that are representative of a patient's breathing. The breathing cycle sensing electronics (399) may coupled to and transmit breathing cycle data to the controller (313). The breathing cycle sensing electronics (399) may also be coupled to the connector (321) for receiving data and/or signals from the lead (320), sub-leads (323a) and (323b) and electrodes (322a) and (322b). Some or all of the functions of the breathing cycle sensing electronics (399) may be performed by software contained within the controller (313).

Atrial defibrillation according to the present disclosure may be done using low-energy (e.g., <2 J), high-voltage (e.g., >80 V), short-duration (e.g., <1000 μs) pulses. Other exemplary energy, voltage and/or pulse duration ranges are set forth in co-owned, co-pending International Patent Application No. PCT/US11/041411, filed on Jun. 22, 2011 and entitled “Pulse Parameters and Electrode Configurations for Reducing Patient Discomfort from Defibrillation,” and International Patent Application No. PCT/US11/044771, filed on Jul. 21, 2011 and entitled “Improved Use of Electric Fields for Reducing Patient,” the disclosures of which are hereby incorporated by reference in their entirety. In other embodiments, a train of two or more pulses may be used. For example, in some embodiments, the IAD (300) may deliver a train of 1-12 pulses. In some embodiments, trains of more than 12 pulses may be delivered.

The IAD (300) may be configured as an atrial defibrillator and pacemaker, an atrial defibrillator and ventricular defibrillator (also known as an implantable cardioverter-defibrillator, or “ICD”) or an atrial defibrillator, ventricular defibrillator and pacemaker. The IAD (300) may be able to monitor, detect and collect data relating to cardiac activity, analyze whether a cardiac condition exists and deliver a defibrillation and/or pacing therapy that best treats the condition. Analyzing the cardiac activity and identifying the existence of a condition may be performed by the controller (313) of the IAD (300), in conjunction with other circuitry and software within the IAD (300). Alternatively, or in addition, cardiac activity analyses and processing may be performed remotely by a medical facility that receives the collected data over the communication link (330). In some embodiments, the IAD (300) may include a patient notification element such as a vibrator, buzzer or other element for alerting a patient when fibrillation has been detected.

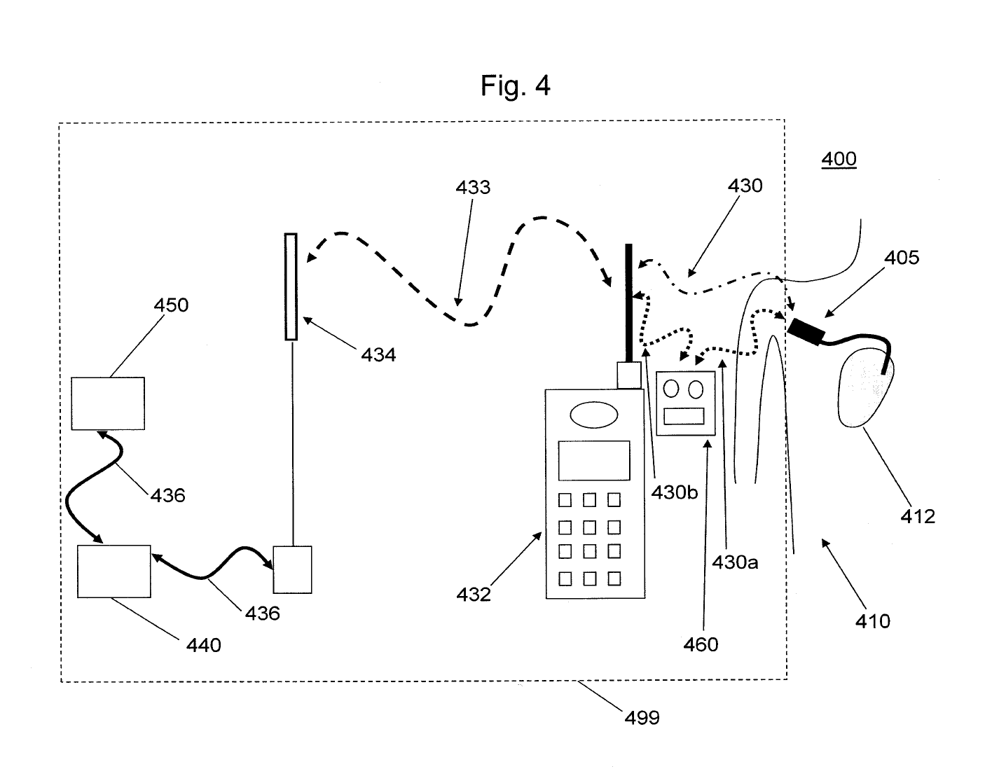

FIG. 4 shows a defibrillation system (400) having an implantable atrial defibrillator (“IAD”) (405) and external components (499) according to the subject matter of the present disclosure. In some embodiments of the system (400), the IAD (405) may be implanted in a patient (410). One or more electrodes (not shown) may be positioned in or around the left and/or right atrium of the heart (412) of the patient (410) for delivering one or more electrical pulses to the heart. The system (400) may include an external communication device (432), an interface device (460) and a server (440), all of which may be in wireless communication with one another. In some embodiments, the IAD (405) may communicate directly with the server (440) or via the external communication device (432) and/or the interface device (460) to, for example, transmit data to the server (440) relating to a possible AF state.

The IAD (405) of the system (400) may communicate with the external communication device (432) via short-range and/or long-range communication. The external communication device (432) may be configured as a two-way communicator capable of transmitting and receiving both data and voice information or, alternatively, the external communication device (432) may be configured to transmit and receive only data or only voice information. In some embodiments, the external communication device (432) may include one or more user inputs, such as a keypad, touch screen, scroll wheel or microphone. Some embodiments of the external communication device (432) may have one or more user outputs, such as a display screen, speaker, vibrating mechanism and/or light-emitting component (e.g., a light-emitting diode). The external communication device (432) may also include a global positioning system (“GPS”) receiver for determining the location of the external communication device (432). The external communication device (432) may be a cellular phone, a smartphone or any other handheld computing device. In some embodiments, external communication device (432) may also be a satellite communication device.

In some embodiments, the IAD (405) may communicate with the external communication device (432) via a communication link (430), as shown in FIG. 4. The IAD (405) may, in some embodiments, communicate with the external communication device (432) via the interface device (460). In some embodiments, the interface device (460) may be an application embedded within the external communication device (432). In some embodiments, the external communication device (432) and/or the interface device (460) may be embedded within the IAD (405) itself, either as software and/or hardware components of the IAD (405). Other embodiments of the present disclosure contemplate the interface device (460) as a separate component in wireless communication with the IAD (405), server (440) and/or external communication device (432). In such embodiments, the interface device (460) may be any shape or size. The interface device (460) may be miniature for discreet placement in or around the heart (412) of the patient (410). The interface device (460), in some embodiments, may be used primarily for providing an interface between the IAD (405) and the external communication device (432) and, thus, may contain no user inputs or outputs. In other embodiments, the interface device (460) may communicate directly with the server (440). The interface device (460) may include user inputs, such as switches or buttons, and user outputs, such as a display screen, speaker(s) and/or vibrating mechanism. In some embodiments, the interface device (460) or external communication device (432) may be used to control the operation of the IAD (405). In some embodiments, the server (440) may use the interface device (460) or external communication device (432) to remotely control the operation of the IAD (405). Communication between the IAD (405) and the external communication device (432) via the interface device (460) may involve using short-range channels. As shown in FIG. 4, the IAD (405) may communicate with the interface device (460) via a short-range channel (430a) and the interface device (460) may communicate with the external communication device (432) via a short-range channel (430b). In some embodiments, the channels connecting the IAD (405), interface device (460) and external communication device (432) may be long-range channels or a combination of short-range and long-range channels.

FIG. 4 also shows that the external communication device (432) may communicate with the server (440) via a long-range communication channel (433). For example, the external communication device (432) may be a mobile phone that communicates with a base station (434) over a long-range communication channel (430), such as a cellular RF channel, and connect to the server (440) over a channel (436). The channel (436) may be a land line, cellular line or other communication channel, such as the Internet. In some embodiments, the external communication device (432) may be a satellite communication device capable of communicating with the server (440) from anywhere around the world. The server (440) may constitute a medical center, hospital and the like, as well as any computers, hospital equipment and human personnel located at any such facility.

In some embodiments, the server (440) may communicate with a rescue team (450) (e.g., a medical team, paramedics and/or an ambulance) over the channel (436) (e.g., land or cellular lines) and direct the rescue team (450) to the location of the patient (410). In some embodiments, the external communication device (432) may communicate directly with the rescue team (450). In some embodiments, once AF has been identified, the IAD (405) may automatically start delivering one or more electrical pulses to the patent (410) to defibrillate the heart. When communication between the IAD (405) and the patient (410) is established and AF is detected, the IAD (405) may initiate communication with the patient (410) via, for example, the external communication device (432) and/or the interface device (460). In some embodiments, medical personnel at the server (440), such as a hospital or other medical establishment, may communicate with the patient (410) and provide instructions and advice (e.g., the patient may be told to breathe slowly or deeply).

FIG. 5 shows a timing sequence (500) for synchronizing the delivery of one or more electrical defibrillation pulses (510) with the expiratory phase (512) of a breathing cycle (514) according to some embodiment of the present disclosure. In some embodiments, one or more R-waves (516) may be separated from each other by 1.0 to 0.3 seconds and the one or more electrical defibrillation pulses (510) may be very short, e.g., lasting less than 150 milliseconds.

In some embodiments, the breathing cycle (514) may depend on the physical and/or mental state of a patient. Heart rates and breathing rates may be strongly influenced by a patient's mental state, e.g., excitement or stress. While heart rate cannot be easily controlled consciously, a patient may partially control his or her breathing rate. For example, a patient may be able to obey commands, such as “breathe in deeply,” “hold your breath” or “exhale fully.” Doing so may change the breathing pattern to establish more favorable conditions for delivering one or more electrical defibrillation pulses. Normal adult breathing rates may range from approximately 12 breaths per minute (i.e., at rest) to 20 breaths per minute (i.e., during exercise) and can reach even higher values during strenuous exercise or disease states. Thus, referring again to FIG. 5, under normal conditions the breathing cycle (514) may be approximately 3-5 seconds long and one or more R-waves (516), separated from each other by 1.0 to 0.3 seconds, may reside within each expiratory phase (512). In some embodiments, a controller (see, e.g., controller (313) in FIG. 3) of a defibrillator according to the present disclosure may track both the R-wave cycle and breathing cycle to determine the time for delivering the one or more electrical pulses (510). Some embodiments may deliver one or more electrical defibrillation pulses (510) immediately after a naturally-occurring R-wave to reduce the probability of inducing ventricular fibrillation.

In some embodiments, the controller (see, e.g., controller (313) in FIG. 3) may choose at least one of the one or more R-waves (516) close to and/or after the peak (524) of the volume capacity of the lungs and within the expiratory phase (512) to deliver one or more electrical defibrillation pulses (510). This could be done by following a few breathing cycles (514) and the one or more R-waves (516), predicting the likely timing of the one or more R-waves (516) close to and/or after the peak (524) of the inspiratory phase (599) and configuring the defibrillator to delivery one or more electrical defibrillation pulses (510) when the one or more R-waves (516) are detected. Because AF defibrillation does not have to occur immediately upon detection (unlike ventricular defibrillation), a patient's breathing pattern may be monitored for some amount of time to more accurately predict when peak inspiration will occur and time a delivery of one or more electrical defibrillation pulses in synchronization with peak inspiration or a short time thereafter, as well as coincide with one or more R-waves (516). Moreover, because respiration is slower than the heartbeat rate, there is sufficient time to detect a coincidence of an R-wave peak and an optimal (or near optimal) timing in the breathing cycle. If the one or more R-waves (516) are not properly detected, and a new inspiration phase (599) has begun, the controller may refrain from producing and/or delivering one or more electrical defibrillation pulses (510) until the next breathing cycle (514).

Some embodiments of the present disclosure may be configured to deliver one or more electrical defibrillation pulses (510) in synchronization with other phases of the breathing cycle (514). For example, at or after the peak (524) of the inspiration phase (599), or during the inhibitory phase of the phrenic nerve, may be determined based on the slope of the breathing cycle (514). In some embodiments, the appropriate phase or instant of the breathing cycle (514) for delivering one or more electrical defibrillation pulses (510) to the heart may be tailored to the patient based on individual testing and/or statistical testing of a group of similar patients.

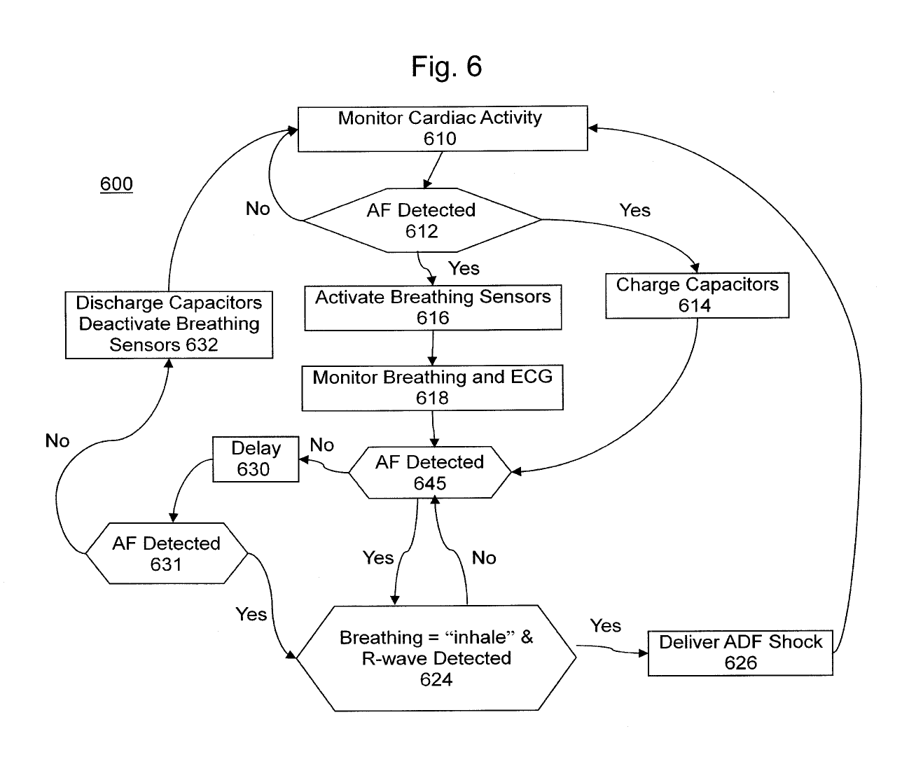

FIG. 6 shows a flow diagram of a method (600) for reducing patient discomfort associated with defibrillation according to some embodiments of the present disclosure. In normal operation, a defibrillator (see, e.g., IAD (300) in FIG. 3) may monitor cardiac activity (610) within a patient. If ventricular fibrillation is detected and the defibrillator can deliver one or more ventricular defibrillation pulses, such pulses will typically be delivered without delay. If atrial fibrillation (AF) is detected (612), one or more breathing sensors (see, e.g., breathing sensors (398) in FIG. 3) may be activated (616) and the breathing cycle sensing electronics (see, e.g., breathing cycle sensing electronics (399) in FIG. 3) may start to monitor (618) the breathing cycle and/or ECG signals (618) of the patient. In some embodiments, one or more high-voltage capacitors in a high-voltage capacitor matrix (see, e.g., high-voltage capacitors and switches matrix (319) in FIG. 3) may be simultaneously charged (614) by a high-voltage generator (see, e.g., high-voltage generator (315) in FIG. 3).

When the one or more high-voltage capacitors are charged and ready to deliver an electrical defibrillation pulse or train of electrical defibrillation pulses (e.g., 1-12 pulses or more than 12 pulses) and AF is still detected (645), the synchronization routine of a controller of the defibrillator (see, e.g., controller (313) in FIG. 3) may determine (624) the correct time to deliver (626) the pulses. If AF is no longer detected (645), the controller of the defibrillator may continue to monitor the breathing cycle and/or ECG signals (618) of the patient for a preset duration (630). If during the time (630) AF is not detected (631), the defibrillator may return to normal operation of monitoring cardiac activity (610) of the patient. In some embodiments, returning to monitoring cardiac activity (610) may involve discharging the capacitors and deactivating the breathing sensors (632). If AF is detected (631) within the preset duration (630), the synchronization routine (624) may be reactivated.

After delivering (626) one or more electrical atrial defibrillation pulses, the defibrillator may return to normal operation to monitor cardiac activity (610). If AF is still detected (612), the operation may repeat. In some embodiments, during subsequent operations, the defibrillator may deliver one or more electrical atrial defibrillation pulses having different parameters, such as higher voltage and/or energy.

Some embodiments of the present disclosure conserve battery power by activating the breathing sensors and/or charging the capacitors only after AF has been detected for some preset time. In some embodiments, activating one or more breathing sensors, charging one or more capacitors and/or delivering an electrical defibrillation pulse may require patient confirmation, for example, using an interface device (see, e.g., interface device (460) in FIG. 4) or an external device (see, e.g., external communication device (434) in FIG. 4). In some embodiments, if patient confirmation is not received within a preset time, a defibrillator may enter an operation mode that delivers one or more electrical defibrillation pulses automatically.

The embodiments set forth in the foregoing description do not represent all embodiments consistent with the subject matter described herein. It is evident that many alternatives, modifications and variations of such embodiments will be apparent to those skilled in the art. As noted elsewhere, these embodiments have been described for illustrative purposes only and are not intended to be limiting. Thus, other embodiments are possible and are covered by the disclosure, which will be apparent from the teachings contained herein. The breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices; that is, elements from one or another of the disclosed embodiments may be interchangeable with elements from another of the disclosed embodiments. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to any of the disclosed embodiments.

1. A defibrillator for defibrillating the heart of a patient, the defibrillator comprising:
an electrode lead system having at least one electrode lead with one or more electrodes;
a controller configured to determine whether a heart is fibrillating and emit a command signal if fibrillation exists;
a voltage generator in communication with the controller and the electrode lead system to produce and discharge one or more electrical pulses to the electrode lead system after receiving the command signal; and
at least one breathing sensor in communication with the controller and configured to collect and transmit information relating to a breathing cycle of the patient to the controller,
wherein the controller processes the information received from the at least one breathing sensor, determines when one or more phases or instants of the breathing cycle of the patient are occurring and emits a command signal to the voltage generator to produce and discharge one or more electrical pulses to the electrode lead system in synchronization with the one or more phases or instants of the breathing cycle of the patient.

2. The defibrillator of claim 1, wherein the defibrillator is positioned outside the patient.

3. The defibrillator of claim 2, wherein the electrode lead system includes at least one external defibrillation electrode positioned outside the patient.

4. The defibrillator of claim 2, wherein the electrode lead system includes at least one internal electrode lead positioned partially in or around the heart of the patient.

5. The defibrillator of claim 2, the electrode lead system further comprising at least one internal electrode lead positioned partially in or around the heart of the patient and at least three external defibrillation electrodes positioned outside the patient.

6. The defibrillator of claim 1, wherein the at least one breathing sensor is positioned on the chest of the patient and contains an electromechanical sensor for detecting chest expansion.

7. The defibrillator of claim 1, wherein the at least one breathing sensor communicates with the defibrillator using wireless means.

8. The defibrillator of claim 1, wherein the at least one breathing sensor collects and transmits information relating to the impedance between at least one electrode of the electrode lead system positioned partially in or around the heart of the patient and at least one electrode of the electrode lead system positioned outside the patient.

9. The defibrillator of claim 8, wherein the at least one electrode of the electrode lead system positioned outside the patient is the defibrillator.

10. The defibrillator of claim 8, wherein the at least one breathing sensor collects and transmits information relating to the impedance between at least one electrode of the electrode lead system positioned partially in or around the heart of the patient and at least one electrode of the electrode lead system positioned outside the patient using a low-voltage, high-frequency signal.

11. The defibrillator of claim 1, wherein the at least one breathing sensor further comprises one or more accelerometers contained within the defibrillator, external to the body of the defibrillator and/or contained within one or more of the at least one electrode lead.

12. The defibrillator of claim 1, wherein the one or more electrical pulses are delivered by the voltage generator to the electrode lead system as a pulse train of one or more sequential electrical pulses.

13. The defibrillator of claim 12, wherein the pulse train consists of up to 12 pulses.

14. The defibrillator of claim 1, wherein the controller emits a command signal to the voltage generator to produce and discharge one or more electrical pulses to the electrode lead system at the peak of the inspiratory phase of the breathing cycle of the patient.

15. The defibrillator of claim 1, wherein the defibrillator is subcutaneously implanted within the patient.

16. The defibrillator of claim 1, wherein the defibrillator is an atrial defibrillator for defibrillating the atria.

17. The defibrillator of claim 1, wherein the electrode lead system includes at least one electrode for sensing atrial fibrillation.

18. The defibrillator of claim 1, the at least one breathing sensor further comprising one or more strain gauges for detecting and measuring movement of the at least one electrode lead caused by breathing action of the patient.

19. The defibrillator of claim 1, wherein the defibrillator analyzes variations in the heart beat of the patient to synchronize the delivery of one or more electrical pulses with one or more phases or instants of the breathing cycle of the patient.

20. The defibrillator of claim 1, wherein the at least one breathing sensor is configured to sense activity of the phrenic nerves of the patient to synchronize the delivery of one or more electrical pulses with one or more phases or instants of the breathing cycle of the patient.

21. The defibrillator of claim 1, wherein the at least one breathing sensor is activated only when fibrillation has been detected and synchronization of one or more electrical pulses with the breathing cycle of the patient is required.

22. The defibrillator of claim 1, wherein the defibrillator automatically produces and delivers ventricular defibrillation pulses to the heart upon detecting ventricular fibrillation.

23. The defibrillator of claim 1, wherein the voltage generator produces and discharges one or more electrical pulses to the electrode lead system in synchronization with the expiratory phase of the breathing cycle of the patient.

24. The defibrillator of claim 23, wherein the voltage generator produces and discharges one or more electrical pulses to the electrode lead system in synchronization with the expiratory phase of the breathing cycle of the patient and immediately after an R-wave of the heart of the patient.

25. The defibrillator of claim 1, wherein the voltage generator produces and discharges one or more electrical pulses to the electrode lead system in synchronization with the inspiratory phase of the breathing cycle of the patient.

26. The defibrillator of claim 25, wherein the voltage generator produces and discharges one or more electrical pulses to the electrode lead system in synchronization with the inspiratory phase of the breathing cycle of the patient and immediately after an R-wave of the heart of the patient.

27. The defibrillator of claim 1, wherein the voltage generator produces and discharges one or more electrical pulses to the electrode lead system during the inhibitory period of the phrenic nerve of the patient based on the information received from the at least one breathing sensor relating to the breathing cycle of the patient.

28-39. (canceled)

40. A heart defibrillation system comprising:
a defibrillator configured to be implanted in a patient, the defibrillator comprising:
an electrode lead system having at least one electrode lead with one or more electrodes;
a controller configured to determine whether a heart is fibrillating and emit a command signal if fibrillation exists;
a voltage generator in communication with the controller and the electrode lead system to produce and discharge one or more electrical pulses to the electrode lead system after receiving the command signal; and
at least one breathing sensor in communication with the controller and configured to collect and transmit information relating to a breathing cycle of the patient to the controller,
wherein the controller processes the information received from the at least one breathing sensor, determines when one or more phases or instants of the breathing cycle of the patient are occurring and emits a command signal to the voltage generator to produce and discharge one or more electrical pulses to the
electrode lead system in synchronization with the one or more phases or instants of the breathing cycle of the patient; and
a communication device disposed outside the patient and configured to communicate with the defibrillator.
